(19) United States
(12) Patent Application Publication
(10) Pub. No.: US 2005/0049476 A1
(43) Pub. Date: Mar. 3, 2005

(54) Human tissue inspection device

(76) Inventor: Si Chang Jang, Seoul (KR)

Correspondence Address: ROBERT W. BECKER & ASSOCIATES, 5300 Sequoia NW, Suite 200, Albuquerque, NM, 87120, US

(21) Appl. No.: 10/641,859

(22) Filed: Aug. 15, 2003

(51) Int. Cl. A61B 5/05

(52) U.S. Cl. 600/407

(57) ABSTRACT

A human tissue inspection device is provided. A human tissue punch has a cylindrical punch body, a punching tip coupled to one end of the punch body to punch human tissue and having a through-hole, a push rod movably disposed in the through-hole of the punching tip to push the human issue captured in the through-hole, a spring placed around the push rod to bias the push rod into the punch body, and a plunger member coupled to a proximal end of the push rod and formed with a shoulder which is to be engaged with the other end of the punch body. An agarose base is made from agarose and paraffin and defined with a plurality of holes so that human tissues of a small size, procured by the human tissue punch, can be respectively inserted and arrayed in the holes.

### BACKGROUND OF THE INVENTION

1. Field of the Invention

The present invention relates, in general, to a human tissue inspection device which is used for procuring human tissue and then enabling human tissue inspection by a microscope and, more particularly, to a human issue inspection device which is constructed to be capable of procuring human tissues of a number of persons, each having a small size, and then arraying the procured human tissues in a single base to enable the human issue inspection by a microscope, so that human tissues of a number of persons can be simultaneously inspected, thereby lowering costs and shortening inspection time.

2. Description of the Related Art

Generally, when checking abnormality of humans in a hospital by screening for various cancers, etc., human issue inspection is conducted in such a way that a portion of human issue is removed from humans and is examined by a microscope.

In the conventional art, in order to conduct human tissue inspection, human tissue of a substantial size is removed using an appropriate implement from a corresponding part of humans for which the human tissue inspection is to be conducted, and then, the removed human tissue is embedded in paraffin. Next, the paraffin including the removed human tissue is cut into a slice, and the slice is placed on a slide made of transparent glass to be inspected by a microscope.

The conventional human tissue inspection is conducted to inspect human tissue of only one person. Therefore, a problem is caused in that, since an inspector must separately perform the above-described procedure for each person for which human tissue inspection is to be conducted, a great deal of time and cost cannot but be incurred.

### SUMMARY OF THE INVENTION

Accordingly, the present invention has been made keeping in mind the above problems occurring in the related art, and an object of the present invention is to provide a human tissue inspection device which is constructed to allow human tissues of a number of persons to be simultaneously inspected, thereby shortening inspection times and reducing inspection costs.

In order to achieve the above object, according to the present invention, there is provided a human issue inspection device comprising: a human tissue punch having a cylindrical punch body, a punching tip threadedly coupled to one end of the punch body to punch and capture human tissue of a small size and defined with a through-hole, a push rod movably disposed in the through-hole of the punching tip to push the human issue captured in the through-hole, a spring placed around the push rod between an intermediate support element coupled to the push rod and a proximal end of the punching tip to bias the push rod into the punch body, and a plunger member having one end which is threadedly coupled to a proximal end of the push rod and the other end which extends beyond the other end of the punch body, the plunger member being formed with a shoulder which is to be engaged with the other end of the punch body; and an agarose base made from agarose and paraffin and defined with a plurality of holes which has the same inner diameter as the through-hole of the punching tip of the human issue punch, so that small samples of human tissues procured by the human tissue punch can be inserted and arrayed in the plurality of holes, respectively.

### BRIEF DESCRIPTION OF THE DRAWINGS

The above and other objects, features and other advantages of the present invention will be more dearly understood from the following detailed description when taken in conjunction with the accompanying drawings, in which:

FIG. 1 is a perspective view illustrating a human tissue punch which serves as one component element of a human tissue inspection device in accordance with an embodiment of the present invention;

FIG. 2 is a sectional view of the human tissue punch shown in FIG. 1;

FIG. 3 is a perspective view illustrating an agarose base which serves as another component element of the human tissue inspection device according to the present invention;

FIG. 4 is a sectional view illustrating a state wherein the agarose base in which human tissues are arrayed is embedded in paraffin; and

FIG. 5 is a plan view illustrating a state wherein a slice obtained by cutting the agarose base into thin pieces is placed on a transparent slide for human tissue inspection by a microscope.

### DETAILED DESCRIPTION OF THE INVENTION

Reference will now be made in greater detail to a preferred embodiment of the invention, an example of which is illustrated in the accompanying drawings. Wherever possible, the same reference numerals will be used throughout the drawings and the description to refer to the same or like parts.

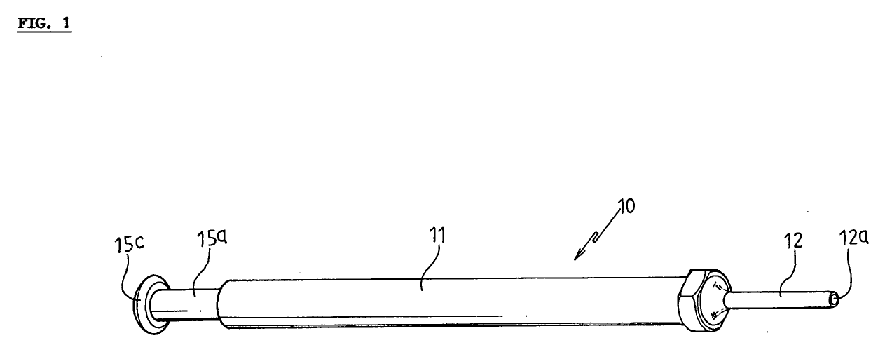

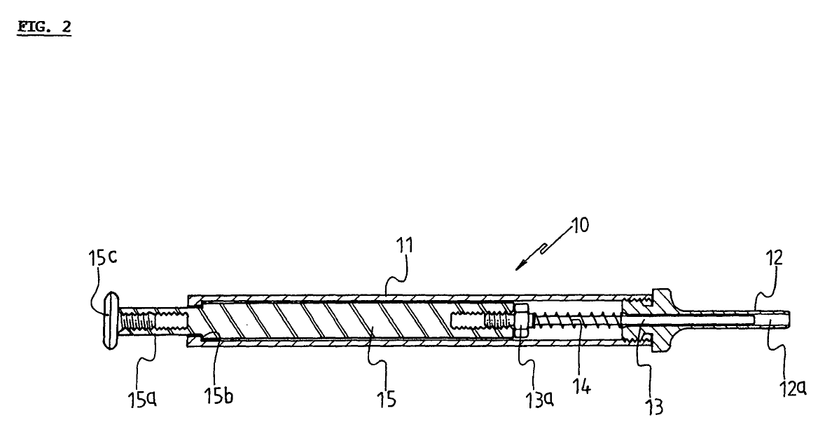

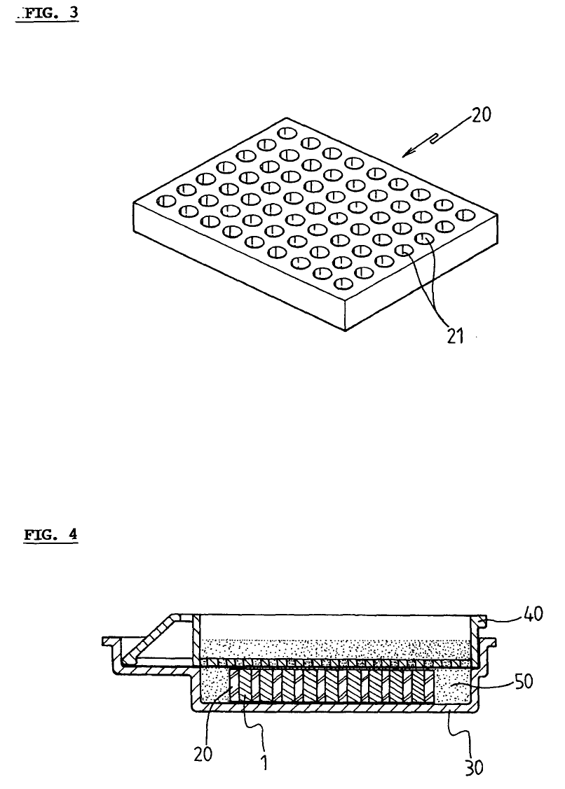

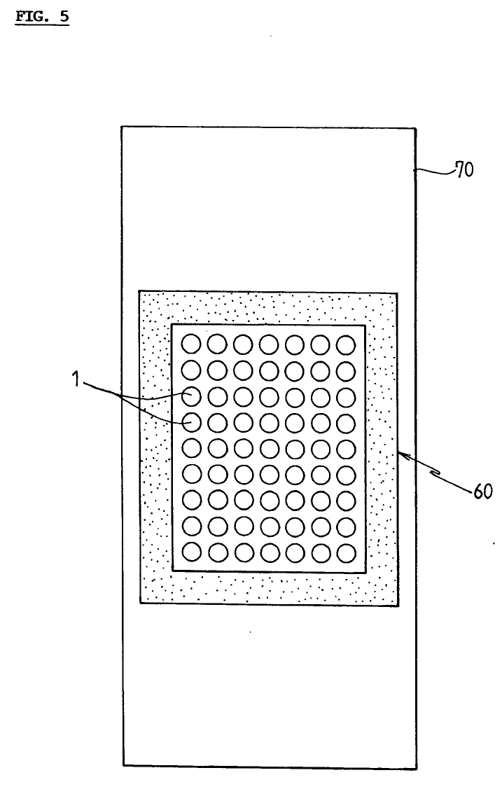

FIG. 1 is a perspective view illustrating a human tissue punch which serves as one component element of a human tissue inspection device in accordance with an embodiment of the present invention. FIG. 2 is a sectional view of the human issue punch shown in FIG. 1. FIG. 3 is a perspective view illustrating an agarose base which serves as another component element of the human tissue inspection device according to the present invention. FIG. 4 is a sectional view illustrating a state wherein the agarose base in which human tissues are arrayed is embedded in paraffin. FIG. 5 is a plan view illustrating a state wherein a slice obtained by cutting the agarose base into thin pieces is placed on a transparent slide for human tissue inspection by a microscope.

A human issue inspection device in accordance with an embodiment of the present invention largely comprises a human tissue punch 10 and an agarose base 20. The human issue punch 10 is used for punching human issue of a small size from large-sized human issue procured from humans. The agarose base 20 is used for fixedly arraying human tissues of the small size punched by the human issue punch 10.

As can be readily seen from FIGS. 1 and 2, the human tissue punch 10 comprises a punch body 11, a punching tip 12, a push rod 13, a spring 14 and a plunger member 15. The punch body 11 has a cylindrical configuration. The punching tip 12 is threadedly coupled to one end of the punch body 11 to punch and capture human issue of a small size. The punch tip 12 is defined with a through-hole 12a. The push rod 13 is movably disposed in the through-hole 12a of the punching tip 12 to push the human issue captured in the through-hole 12a to the outside. The spring 14 is placed around the push rod 13 between an intermediate support element 13a coupled to the push rod 13 and a proximal end of the punching tip 12, to bias the push rod 13 into the punch body 11. The plunger member 15 has one end which is threadedly coupled to a proximal end of the push rod 13 and the other end 15a which extends beyond the other end of the punch body 11. The plunger member 15 is formed with a shoulder 15b which is to be engaged with the other end of the punch body 11.

A distal end of the punching tip 12 is formed with a sharp edge, similar to that of a knife, so that the punching tip 12 can be easily penetrated into the human issue. In this regard, it is to be noted that a variety of punching tips 12 having through-holes of different inner diameters can be appropriately used as occasion demands.

The proximal end of the push rod 13 is formed with an external thread and one end of the plunger member 15 is defined with an internal thread, so that the push rod 13 and the plunger member 15 are threadedly coupled with each other. The intermediate support element 13a is coupled adjacent to the proximal end of the push rod 13. The intermediate support element 13a has a nut shape so that the push rod 13 and the plunger member 15 can be threadedly coupled to each other in an easy manner.

The other end 15a of the plunger member 15 has a small diameter to project beyond the other end of the punch body 11, by which the shoulder 15b is formed adjacent to the other end of the plunger member 15. The other end 15a of the plunger member 15 is defined with a threaded groove, and a knob 15c is threadedly coupled into the threaded groove. The knob 15c is shaped so that it can be pushed into and pulled out of the punch body 11 with fingers.

The agarose base 20 is made from agarose and paraffin. As shown in FIG. 3, the agarose base 20 is defined with a plurality of holes 21 which have the same inner diameter as the through-hole 12a of the punching tip 12 of the human tissue punch 10, so that small samples of human tissues procured by the human tissue punch 10 can be inserted and arrayed in the plurality of holes 21, respectively.

Hereafter, the operations of the human tissue inspection device according to the present invention, constructed as mentioned above, will be described.

First, a procedure for punching human tissue of a small size from large-sized human tissue procured from humans, using the human tissue punch 10, will be described. If an inspector grasps and presses the punch body 11 of the human tissue punch 10 against the large-sized human tissue, as the large-sized human tissue is punched, the human tissue 1 of a small size is inserted into the through-hole 12a of the punching tip 12. Then, in a state wherein the distal end of the punching tip 12 is aligned with one hole 21 of the agarose base 20, by pressing the knob 15c threadedly coupled to the plunger member 15 using the thumb, the human tissue 1, which is inserted into the through-hole 12a of the punching tip 12, is pushed to the outside by a distal end of the push rod 13 and introduced into the corresponding hole 21 of the agarose base 20. Then, by releasing the plunger member 15, the push rod 13 is returned to its original position by the elasticity of the spring 14.

By repeatedly conducting the above-described tasks, human tissues of a number of persons are respectively arrayed in the plurality of holes 21 of the single agarose base 20. Thereafter, the agarose base 20 having the arrayed human issue is embedded in paraffin.

In order for this embedding, as shown in FIG. 4, the agarose base 20 is received in an agarose base mold 30, and a cassette 40 which is also defined with a plurality of holes is placed on the agarose base 20. Thereupon, solid paraffin is put on the cassette 40, and heat is applied to the solid paraffin.

By doing this, as the paraffin is melted, the melted paraffin flows downward through the holes of the cassette 40 to surround the agarose base 20 as shown in FIG. 4. At this time, the agarose base 20 is also partially melted to be fused with the paraffin 50.

In this state, as the paraffin 50 is cured, the cassette 40 and the agarose base 20 are integrated with each other by the paraffin 50. If the cassette 40 is lifted, the agarose base 20 having arrayed therein the human tissues 1 is also integrally lifted.

Then, in a state wherein the cassette 40 is secured to a slicer which serves as a machine for cutting a sample into thin pieces, the human tissue and the paraffin 50 are cut into a plurality of very thin slices 60. After chemically treating a slice 60, as shown in FIG. 5, the slice 60 is placed on a slide 70 made of transparent glass. In this way, human tissue inspection is conducted by a microscope.

Therefore, since human tissues 1 of a number of persons are held arrayed on the slide 70, the inspector can simultaneously conduct human tissue inspection for the human tissues 1 of a number of persons.

The human tissue inspection device according to the present invention is not limited to the inspection of human tissue and can be used for inspection of animal tissue.

As apparent from the above description, the human tissue inspection device according to the present invention provides advantages in that, since human tissues of a number of persons are respectively procured using a human tissue punch and then arrayed in a single agarose base, human tissues of a number of persons can be simultaneously inspected, whereby an inspection time can be shortened and an inspection cost can be reduced.

Although a preferred embodiment of the present invention has been described for illustrative purposes, those skilled in the art will appreciate that various modifications, additions and substitutions are possible, without departing from the scope and spirit of the invention as disclosed in the accompanying claims.

Therefore, the present invention is, of course, in no way restricted to the specific disclosure of the specification and drawings, but also encompasses any modifications within the scope of the appended claims.

1. A human tissue inspection device comprising:
a human issue punch having a cylindrical punch body,
a punching tip threadedly coupled to one end of the punch body to punch and capture human tissue of a small size and defined with a through-hole,
a push rod movably disposed in the through-hole of the punching tip to push out human issue captured in the through-hole,
a spring placed around the push rod between an intermediate support element coupled to the push rod and a proximal end of the punching tip to bias the push rod into the punch body,
a plunger member having one end which is threadedly coupled to a proximal end of the push rod and the other end which extends beyond the other end of the punch body, the plunger member being formed with a shoulder which is to be engaged with the other end of the punch body, and
an agarose base made from agarose and paraffin and defined with a plurality of holes which have the same inner diameter as the through-hole of the punching tip of the human tissue punch, so that human tissues of the small size procured by the human issue punch can be inserted and arrayed in the plurality of holes, respectively.
